(19) United States
(12) Patent Application Publication
(10) Pub. No.: US 2007/0122816 A1
(43) Pub. Date: May 31, 2007

(54) Microarray comprising qc probes and method for fabricating the same

(75) Inventors: Hee Kyung Park, Seoul (KR); Cheol Min Kim, Busan-city (KR); Hyun Jung Jang, Busan-city (KR)

Correspondence Address: GREENLEE WINNER AND SULLIVAN P C, 4875 PEARL EAST CIRCLE, SUITE 200, BOULDER, CO, 80301, US

(73) Assignees: Hee Kyung Park, Seoul (KR); Cheol Min Kim, Busan-city (KR)

(21) Appl. No.: 10/597,718

(22) PCT Filed: Aug. 2, 2004
(86) PCT No.: PCT/KR04/01939

§ 371 Date: Sep. 14, 2006

(30) Foreign Application Priority Data
Feb. 4, 2004 (KR) 10-2004-0007237

(51) Int. Cl. C12Q 1/68; C07H 21/04

(52) U.S. Cl. 435/6; 536/24.3; 536/25.32

(57) ABSTRACT

A quality control (QC) probe for inspecting a quality of a microarray, a method for fabricating a microarray in which the QC probe and a target probe are immobilized on a support, and a method for inspecting the quality of a microarray using the QC probe are provided. More particularly, a method for fabricating a microarray by mixing a QC probe labeled with a fluorescent material and a target probe at a certain ratio and immobilizing the mixture on a support of a microarray, a method for inspecting the quality of a microarray including identifying the immobilization state of probes by scanning a fluorescent signal produced by a fluorescent material before or after a hybridization reaction of a target probe and a target product using the prepared microarray, and a QC probe used for inspecting the quality of a microarray are provided. The QC probe can be used to identify whether or not each probe is immobilized on a support of a microarray, shape and concentration of the immobilized probe, and a bonding reaction or a hybridization reaction of a target probe and a target product. When using the microarray including the QC probe in a hybridization reaction, a reliability of experimental procedures and result analysis using the microarray can be improved. In addition, the use of a target probe having a QC function can simplify the process of fabricating a microarray.

### TECHNICAL FIELD

The present invention relates to a fluorescence-labeled, quality control (QC) probe which is used in a microarray, a method for fabricating a microarray including the same, and a method for inspecting a quality of a microarray using the QC probe included in the microarray. More particularly, the present invention relates to a method for fabricating a microarray by mixing a quality control probe (hereinafter, referred to as “QC probe”) labeled with a fluorescent material and a probe reacting with a target product (hereinafter, referred to as “target probe”) at a certain ratio and spotting the mixture on a support of a microarray, a method for fabricating a target probe having a QC function by labeling with a fluorescent dye at any position in the base sequence of the target probe, a method for inspecting the quality of a microarray including identifying the immobilization state of probes by scanning fluorescent signal produced by a fluorescent material before or after a hybridization reaction of a target probe and a target product using the prepared microarray, and a QC probe used for inspecting the quality of a microarray.

### BACKGROUND ART

A microarray is a bio-chip in which a number of biomolecules, such as DNA, protein, lectins, cell, etc., are arranged and immobilized by uniformity on a solid supports of glass, silicone, or nylon. The presence of a disease-related gene or protein, etc. may be detected by analyzing a bonding pattern between a target product to be analyzed and the immobilized biomolecule, i.e., a probe. Microarrays are divided into DNA chips in which DNA is immobilized and protein chips in which protein is immobilized according to the kind of immobilized probe. DNA chips may be divided into a pin microarray chip, an inkjet chip, a photolithography chip, and an electronic array chip according to a method of immobilizing DNA on a surface of a chip.

In a biochip, a probe including genetic information, such as oligonucleotide, cDNA, protein, etc., is immobilized on a surface of a support. A fluorescent material is bound with DNA, cDNA or DNA proliferated by a polymerase chain reaction (PCR), etc. in a sample. The biomolecule is allowed to bind only to a probe having a complementary sequence through a hybridization reaction, and the bonding aspect is qualitatively and quantitatively analyzed [Duggan D. J., Bittner M., Chen Y., Meltzer P. and Trent J. M., Expression profiling using CDNA microarrays, Nat. Genet. Supp. 21:10-14, 1999, Vivian G. Cheung, M. Morley, F. Aguilar, A. Massimi, R. Kucherlapati, G. Childs, Nature genetics, 1999, 21: 15-19]. The results of hybridization reaction aspect of the target product and the target probe are significantly affected by whether or not the probe including oligonucleotide, etc., is immobilized on the support and the concentration of the immobilized probe. Therefore, identification regarding whether the probe (spot) is immobilized or not and the concentration of the immobilized probe (spot) is very important. Also, the hybridization reaction performed without identification regarding whether or not all target probes are immobilized significantly affects the results of qualitative and quantitative analysis for the bonding aspects of the target probe and the target product.

In the biochip, the spotting of a prepared target probe on a support using a spotter, a hybridization reaction of a target product and the target probe, and analysis using a scanner are performed. Since it is impossible to manually inspect each chip during these processes, quality control of a number of microarray elements is very important in the fabrication of a microarray [V. Chizhikov, M. Wagner, A. Ivshina, Y. Hoshino, A. Z. Kapikian, and K. Chumakov, Detection and genotyping of human group a Rotaviruses by oligonucleotide microarray hybridization, Journal of Clinical Microbiology, 40(7):2398-2407, 2002].

Thus, to improve the reliability for result analysis of a microarray, it is necessary to identify the quality of the microarray before a hybridization reaction. Brown et al. in Stanford University developed a method of inspecting the uniformity of a DNA probe spotting and whether a surface of glass is damaged by detecting light scattered by a salt present with a DNA probe spotted on the surface of glass using a laser scanner. However, this method can inspect DNA spots only immediately after spotting and cannot inspect the quality of DNA spots after a DNA chip has been fabricated since the salt placed in the DNA spots is removed after immobilizing and washing.

Recently, in a quality control method for the fabrication of a microarray, a microarray is dyed with a dye such as SYBR green II emitting fluorescent light due to a specific affinity for single-stranded DNA, and then the emitted fluorescent light is analyzed using a laser scanner. This method is used for the evaluation of qualities, on a surface of a microarray support, integrity, and homogeneity of each spot and the like [Battaglia C, Salani G, Consolandi C, Bernardi L R, and De Bellis G., Analysis of DNA microarrays by non-destructive fluorescent staining using SYBR green II, Biotechniques 29(1):78-81, 2000]. However, in this method, a complicated process for completely removing the fluorescent dye should be preformed in order to use the microarray in a hybridization reaction of a main experiment. Meanwhile, in the method of the present invention, a quality of a microarray can be identified only by a scanning process before entering the main experiment. Further, although it is reported that the microarray used for the quality control using SYBR green II etc, can be reused after obtaining results, reuse is inefficient since the lowering of the efficiency of the hybridization reaction is 12% or more, and this lowers the efficacy of the product.

In addition, for the purpose of a quality control during the fabrication of a microarray and hybridization reaction, both a target probe and a reference oligonucleotide QC probe are immobilized on a support, a synthetic oligonucleotide, which is complementary to a QC probe and is labeled with a fluorescent material having a different wavelength from that of the fluorescent material labeled to the target probe, is mixed with the target product, and reacted with the microarray, and information regarding distributions of the target probe and the reference oligonucleotide after the hybridization reaction can be obtained using two different wavelengths after the hybridization reaction [V. Chizhikov, M. Wagner, A. Ivshina, Y. Hoshino, A. Z. Kapikian, and K. Chumakov, Detection and genotyping of human group a Rotaviruses by oligonucleotide microarray hybridization, Journal of Clinical Microbiology, 40(7):2398-2407, 2002]. However, in this method, quality control is possible only after dyeing of the produced microarray, hybridization reaction, etc., are performed, and the synthetic oligonucleotide complementary to QC probe is separately required in addition to the target product. Also, accurate quality control for each microarray is impossible and the same experimental procedures as the main experiment are performed for QC. Thus, this method is inefficient.

As a result of efforts to overcome drawbacks, such as inefficiency, in the conventional quality control method when fabricating a microarray, the inventors found that quality of a microarray can be economically, rapidly and accurately inspected when a target probe and a fluorescence-labeled QC probe are mixed and spotted or only a target probe having a QC function by adding a fluorescent dye to the target probe is spotted, and then each probe spotted in a slide is subject to quality identification using a scanner before the hybridization reaction and is used in a main experiment, thereby completing the present invention.

### BRIEF DESCRIPTION OF THE DRAWINGS

FIG. 1 is a design of a quality control (QC) probe used to identify the immobilization of probes and a hybridization reaction;

FIG. 2 illustrates probes immobilized on a support after mixing a QC probe and a target probe;

FIG. 3 is a design of a QC probe simultaneously acting as a target probe as an embodiment of the present invention, in other words, a target probe having a QC function;

FIGS. 4A through 4C are results analyzing a slide with a scanner after mixing a QC probe and a target probe at a certain ratio and immobilizing the mixture in the slide and before washing the slide and FIG. 4D is a schematic diagram of an arrangement of target probes spotted on each slide;

FIG. 5 is the result analyzing a slide with a scanner after immobilizing only target probes in the slide without using a QC probe and before washing the slide;

FIGS. 6A through 6C are results analyzing a slide with a scanner after immobilizing QC probes and target probes in the slide and washing the slide, wherein 1) is the result of analyzing the slide with scanner after washing and before a hybridization reaction; and 2) illustrates the slide analyzed after a hybridization reaction, and FIG. 6D is a schematic diagram of an arrangement of target probes spotted on each slide; and

FIGS. 7A and 7B are results of analyzing a probe in which a fluorescent dye is introduced into a spacer of a target probe or the internal position of a target base sequence, wherein 1) is the result of analyzing the probe at a wavelength (532 nm) of TAMRA labeled for QC after immobilizing the probe on a support and washing it; and 2) is the result of analyzing whether hybridization with the target product has occurred after a hybridization reaction.

### DETAILED DESCRIPTION OF THE INVENTION

### Technical Goal of the Invention

The present invention provides a quality control (QC) probe with a fluorescence-label for inspecting the quality of a microarray.

The present invention also provides a new microarry containing the fluorescence-labeled QC probe, which can easily perform a quality inspection, and a method for fabricating the same.

The present invention also provides an economical, rapid and accurate method of inspecting the quality of a microarray, which can identify an immobilization of probes of the microarray and a bonding (hereinafter, also referred to as “hybridization”) reaction with the target product using the QC probe contained in the prepared microarray.

### Disclosure of the Invention

According to an aspect of the present invention, there is provided a quality control (QC) probe for inspecting the quality of a microarray, wherein an oligonucleotide having a complementary sequence to a base sequence of a target product of the microarray or having any base sequence is labeled with a fluorescent material and the fluorescent material has an excitation/emission wavelength different from that of a fluorescent material labeled in the target product.

In the present invention, the fluorescent material labeled in the QC probe has an excitation/emission wavelength different from that of the fluorescent material labeled in the target product. Thus, even after identifying the spotted probe in the microarray by analyzing the QC probe labeled with the fluorescent material at a specific wavelength before a main experiment, for example, for diagnosis or research, hybridization reactions with the probe arrayed on the support can be identified in the main experiment without spectral interference.

In the present invention, the fluorescent material may contain at least one material selected from materials listed in Table 1 but is not limited thereto, and any fluorescent material having a different wavelength from the target product may be used. For example, when Cy5 fluorescent material is used to identify the presence of a bonding reaction of the target product and the target probe, the QC probe may use a fluorescent material having different wavelength, such as Cy3 or TAMRA.

TABLE 1
Fluorescent material used for the quality
control in the fabrication of a microarray

| | Excitation | Emission | Emission |
|---|---|---|---|
| Fluorescent material | (nm) | (nm) | filter |
| Pyrene | 340 | 376, 395 | 430 |
| Cyanine 2 | 489 | 506 | 508 |
| GFP | 488 | 507 | 508 |
| Calcein | 494 | 517 | 522 |
| FITC | 494 | 518 | 522 |
| Alexa 488 | 490 | 520 | 522 |
| FAM | 490 | 520 | 522 |
| Fluorescein Chlorotriazinyl | 492 | 514 | 522 |
| Fluorescein | 494 | 517 | 522 |
| Rhodamine 110 | 500 | 525 | 522 |
| Oregon Green | 500 | 524 | 522 |
| Magnesium Green | 506 | 531 | 530 |
| Calcium Green | 506 | 533 | 530 |
| JOE | 524 | 550 | 549 |
| Cyanine 3 | 550 | 570 | 570 |
| Tetramethylrhodamine | 550 | 570 | 570 |
| TRITC | 547 | 572 | 570 |
| TAMRA | 560 | 582 | 578 |
| Rhodamine Phalloidin | 550 | 575 | 578 |
| Pyronin Y | 555 | 580 | 578 |
| Lissamine | 570 | 590 | 592 |
| ROX | 588 | 608 | 614 |
| Calcium Crimson | 590 | 615 | 614 |
| Texas Red | 595 | 615 | 614 |
| Nile Red | 549 | 628 | 630 |
| Cyanine 5 | 649 | 670 | 670 |
| Thiadicarbocyanine | 651 | 671 | 670 |

In the present invention, the QC probe may be an oligonucleotide having a complementary sequence to a base sequence of the target product like probes attached to conventional general supports or having any sequence. The fluorescent material may be labeled at one or more positions in a base sequence of the oligonucleotide, and any position of the probe, such as 3′ end, 5′ end or the internal position of the QC probe may be labeled.

In the present invention, the QC probe may or may not have a spacer between a base sequence of the probe and the fluorescent material. In this case, the spacer may be any molecule capable of linking the fluorescent material to the QC probe without affecting hybridization, and examples thereof include C-3 linker, C-6 linker, C-6 TFA linker, C-5 amino modifier, C-12 linker, Amino dT C2 linker, Amino dT C6 linker, 3′ branched amino CPGs, 3′ C3 amino modifier, 3′ C7 amino modifier, 5″ Thiol C-2 linker, 5′ Thiol C-6 linker, 5′ Thiol C-6 S—S, 3′ Thiol C3, and the like. Also, the QC probe may or may not have a spacer between the probe and the support. In this case, the spacer may be any molecule capable of linking the probe to the support without affecting hybridization, and examples thereof include C-3 linker, C-6 linker, C-6 TFA linker, C-5 amino modifier, C-12 linker, Amino dT C2 linker, Amino dT C6 linker, 3′ branched amino CPGs, 3′ C3 amino modifier, 3′ C7 amino modifier, 5″ Thiol C-2 linker, 5′ Thiol C-6 linker, 5′ Thiol C-6 S—S, 3′ Thiol C3, and the like.

In the present invention, the QC probe is not separately used and the target probe having fluorescent material whose excitation/emission wavelength is different from that of the fluorescent material labeled to a target product, may be used as a QC probe. Hereinafter, such a QC probe is referred to “QC probe simultaneously acting as a target probe”. In this case, the QC probe has a complementary sequence to the base sequence of the target product, and thus acts as a target probe. In other words, immobilization of probes and a hybridizaton reaction of the target product and the target probe may be simultaneously identified.

In the QC probe simultaneously acting as a target probe, the fluorescent material may be labeled at a position of 3′ end, 5′ end or the internal position of the target probe and the fluorescent material may be labeled at the spacer of the target probe or in the internal position of the target base sequence.

According to another aspect of the present invention, there is provided a method for fabricating a microarray, the method including immobilizing a QC probe labeled with a fluorescent material and a target probe mixed at a certain ratio on a support of the microarray or immobilizing a QC probe simultaneously acting as a target probe (in other words, a target probe having QC function) alone on the support of the microarray, and a microarray prepared by the method and containing the QC probe labeled with a fluorescent material.

In the method, the mixing ratio of the QC probe and the target probe is not limited as long as the QC probe and the target probe are uniformly mixed, and thus a relative ratio of the QC probe to the target probe is the same. The target probe and the QC probe may be mixed at a ratio of 30-50 pmol of the target probe per 1-5 pmol of the QC probe to perform the function of the QC probe without interfering with the function of the target probe.

According to another aspect of the present invention, there is provided a method for inspecting the quality of a microarray, the method including identifying the immobilization state of probes by scanning fluorescence signals produced by a fluorescent material before or after a hybridization reaction of a target probe and a target product using a microarray containing the QC probe labeled with the fluorescent material.

In the present method, only the microarray in which quality inspection for each of the spots in the microarray has been completed by scanning the fluorescent signal produced by the fluorescent material of the QC probe prior to a hybridization reaction may be used in a main experiment. Thus, an accurate qualitative and quantitative analysis of the bonding aspects of the target probe and the target product may be obtained, and thus, an economical, rapid and accurate method for inspecting the quality of the microarray is provided. Also, the present invention can save time by scanning fluorescent signals produced by the fluorescent material of the QC probe with the fluorescent signals produced by the fluorescent material of the target product at a different wavelength respectively.

In a hybridization reaction using a conventional probe attached to a support, the probe is designed only to have a complementary sequence to a target base sequence. However, in the present invention, a target probe to be examined and a QC probe having an attached fluorescent material are provided together to rapidly and accurately identify a hybridization reaction and an immobilization state of the probe on the support and whether a problem is occurred during spotting.

In the present invention, the target probe may be any biomaterial such as cDNA, oligonucleotide, peptide, or protein according to the type of a microarray. Examples of the support include, but are not limited to, glass, membrane, gold, gel-covered surface, semiconductive chip, silicon, polymer, and the like conventionally used in the fabrication of a microarray. The support may be coated with a silanization and polymer matrix, and the like, and the fluorescent signal may be detected with a laser scanner, a CCD scanner, and the like commercially used.

In the present invention, the microarray includes a combination of the target probe with the QC probe labeled with the fluorescent material or the QC probe simultaneously acting as the target probe (in other words, the target probe having the QC function) alone in each spot on the support. Thus, in relation to immobilization of the probe in the fabrication of a microarray, the quality of each probe of the microarray may be rapidly and accurately determined. When using the QC probe mixed with the target probe, the QC probe and the target probe may have a mixing ratio according to an object without limitation. However, a high concentration of the QC probe labeled with a fluorescent material may affect the intensity of fluorescence from the fluorescent material attached to the target base sequence during a hybridization reaction with the is target probe. When using the QC probe labeled with a fluorescent material in a high concentration, the production costs are increased. Thus, the concentration of the QC probe labeled with the fluorescent material should be as low as possible. Also, when using the QC probe simultaneously acting as a target probe alone, the target probe is labeled with a fluorescent dye so as to simultaneously act as the target probe and as the QC probe for quality control.

According to another aspect of the present invention, there is provided a microarray containing the QC probe together with a target probe in one spot.

In the present invention, the microarray may contain different target probes according to the use of the microarray, and whether the probe is immobilized or not and states such as shape, concentration and the like, of a spot, and hybridization reaction with a target product may be identified.

The present invention will be described in greater detail with reference to the following examples using a microarray for differentiating Mycobacteria strains. The following examples are for illustrative purposes and are not intended to limit the scope of the invention.

### Effect of the Invention

According to a method for inspecting the quality of a microarray using a quality control (QC) probe labeled with a fluorescent material of the present invention, the immobilization of a target probe to be examined and the shape and condition of spot such as uniformity, noncircular spots, irregular shapes, dust, doughnut phenomenon of each probe in the microarray may be analysed through one spotting. A QC probe can simply and accurately inspect the target probe on a support as well as a hybridization reaction. In other words, a conventional probe for a hybridization reaction is designed to have complementary sequence to the target base sequence to be used only for a hybridization reaction, but the probe designed in the present invention can inspect a probe attached to the support as well as a general hybridization reaction by being labeled with a fluorescent dye. In the present invention, immobilization of the target probe and a hybridization reaction with a target product can be identified using only the target probe labeled with a fluorescent dye, and thus, the inspection process can be simply performed. Since the QC probe is labeled with a fluorescent material having a different wavelength from that of the fluorescent material for a hybridization reaction, the identification of the probe after a hybridization reaction is possible. Therefore, problems in experiment procedures may be checked, and thus results can be more accurately analysed. Thus, the present invention can be economically, rapidly and accurately used in the fabrication of a microarray and the inspection of the quality thereof, for identifying immobilization and the state of probes in the microarray and investigating various effects on a hybridization reaction.

### BEST MODE FOR CARRYING OUT THE INVENTION

### Example 1

### Isolation of Genome DNA of Mycobacteria Strain

Mycobacteria standard strains were obtained from Korean Collection for Type Culture (KCTC) and American Type Culture Collection (ATCC) and InstaGene matrix (available from Bio-Rad Co., USA) was used to extract DNA thereof.

The strain to be used in the present Example was cultured in a solid culture medium (Ogawa medium), and then was placed and floated in a 1.5 ml tube containing 200 μl of InstaGene matrix. It was reacted at 56° C. for 30 minutes and thoroughly mixed for 10 seconds. Then, the mixture was heated at 100° C. for 8 minutes and thoroughly mixed for 10 seconds. The mixture was centrifuged at 12,000 rpm for 3 minutes and the separated supernatant was transferred to a new tube. It was used as template DNA for a polymerase chain reaction (PCR).

The following standard strains were used:

M. tuberculosis H37Rv (ATCC 27294)

M. fortuitum (ATCC 6841)

M. avium (ATCC 25291)

M. intracellulare (ATCC 13950)

M. kansasii (ATCC 12478)

M. chelonae (ATCC 35752)

M. abscessus (ATCC 19977)

M. gordonae (ATCC 14470)

M. vaccae (ATCC 15483)

M. xenopi (ATCC 19250)

M. smegmatis (ATCC 21701)

M. genavense (ATCC 51233)

M. malmoense (ATCC 29571)

M. simiae (ATCC 25275)

M. marinum (ATCC 927)

M. ulcerans (ATCC 19423)

M. gastri (ATCC 15754)

M. terrae (ATCC 15755)

### Example 2

### A Target Probe for Differentiating Mycobacteria Strain

An oligonucleotide probe used in the present invention was prepared by synthesizing a probe having a dT spacer having 5′-Amino-Modifier C6-15 bases at 5′ end and 15-25 base sequences using Perkin Elmer DNA Synthesizer (available from Perkin Elmer DNA Synthesis, USA) and PAGE purifying the resultant [Korean Patent Application No. 2001-0062125, entitled “Microarray comprising probes for Mycobacteria strain genotyping, M. Tuberculosis strain differentiation, and antibiotic-resistant strain detection, detection method using the microarray, and diagnostic kit, filed on Oct. 09, 2001 by Kim, Cheol-Min and Park, Hee-Kyung in S J Hightech Co., LTD.] The probe fabricated in the present invention was as follows

TABLE 2

Base sequence of the target probe

| | | | SEQ |
|---|---|---|---|
| | | | ID |
| Identified strain | Probe | Base sequence | No |
| Mycobacteria | ITSF-2 | GCTTTCTAAGGAGCACCACG | 1 |
| | ITSF-3 | GCTTTCTAAGGAGCACCATT | 2 |
| | Mycom2R | TGGATAGTGGTTGCGAGCAT | 3 |
| TB complex | MTB-02 | TGGTGGGGCGTAGGCCGTGA | 4 |
| M. avium-M. | MAC-03 | CTCGGTCGAACCGTG | 5 |
| intracellulare | | | |
| (MAC) | | | |
| M. fortuitum | FOR-04 | CAAACTTTTTTGACTGCCAG | 6 |
| M. chelonae | CHE-04 | GTAGTCGGCAAAACGTCGGA | 7 |
| Internal control | INT-01 | CAGTTATATGGATGATG | 8 |

### Example 3

### Fabrication of a QC Probe Labeled with a Fluorescent Dye

A fluorescent material used for labelling a QC probe was selected to have a different wavelength from that of the fluorescent material used for the target probe. For example, when selecting Cy5 using an emission filter of 670 nm to identify a bonding reaction of a target product and the target probe in an oligonucleotide chip, a fluorescent material having a different wavelength from that of Cy5 in a main experiment, such as Cy3 or tetramethylrhodamine (TAMRA) having a wavelength at 570 nm was used as the fluorescent material labelling the QC probe. In the case of the cDNA chip, when using Cy3 and Cy5 in the target probe, a fluorescent material having a third wavelength different from that of the fluorescent materials was used in the synthesis of the QC probe for a quality inspection.

In the Example, Cy5 fluorescent material was used to dye the target product and TMARA fluorescent material was used to fabricate the QC probe, thereby designing 5′-Amino-Modifier C6 20-50 mer—TAMRA QC probe. The base sequence of the QC probe used in this Example was as follows.

(SEQ ID NO. 9)

5′-TTT TTT TTT TTT TTT TTT TTT TTT TTT TTT-TAMRA-
3′

(SEQ ID NO. 10)

5′-TTT TTT TTT TTT TTT Tgg Tgg ggT gTg gTg TTT gA-
TAMRA-3′

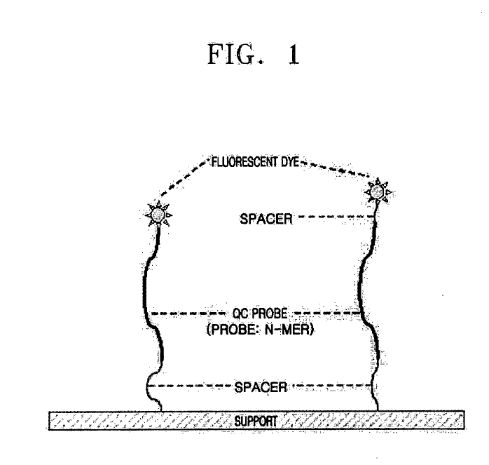

Referring to FIG. 1, a probe having a fluorescent material directly on the same base sequence as the target probe or any base sequence on a support, in which space is placed between the base sequence and the support, or a probe having a spacer placed between the base sequence and the fluorescent material may be used as the QC probe.

### Example 4

### Attachment of Probe to Support

Each of target probes fabricated in Example 2 was diluted to a concentration of 30-50 pmol and transferred to 96-well microplates. The QC probe (prepared in Example 3) diluted to a concentration of 1-5 pmol and Micro-spotting solution or 3× SSC solution were added to each well and thoroughly mixed.

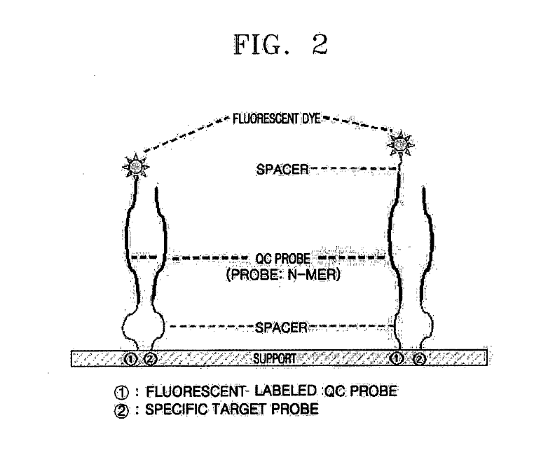

In the present experiment, 1 pmol of the QC probe and 30 pmol of the target probe were mixed, but the mixing ratio may be varied without affecting the results. The mixed probe was attached to a support such as slide glass or membrane using PLXSYS 7500 SQXL Microarrayer (available from Cartesian Technologies, USA) as illustrated in FIG. 2. Two spots per one kind of probe were attached to the support and then left in a slide box at room temperature for about 24 hours or in a dry oven at 50° C. for about 5 hours to immobilize the spots on the surface of the support.

### Example 5

### Preparation of a Target Product

To amplify a target product for differentiating Mycobacteria strains, a target site 300-500 bp of the strain isolated in Example 1 was selectively amplified by performing a PCR method using a primer labeled with biotin. In this Example, biotin-labeled primers ITSF-2, ITSF-3, and Mycom2R were used.

The PCR was performed by heating at 94° C. for 3 minutes to sufficiently denature, reacting 30 times at 94° C. for 1 minute, at 62° C. for 1 minute, and at 72° C. for 1 minute, and then extending at 72° C. for 10 minutes in Perkin-Elmer Cetus Thermocycler Model 9600. This method was described in detail in J. Clin. Microbiol. 38: 4080-4085 (2000) by Park H. K. et al., which is incorporated herein by reference.

### Example 6

### Investigation of Quality Control for Immobilization of Probes

Since whether the probe was immobilized in the slide during immobilizing before washing and the immobilization state was important in the analysis of the results and the state of the spots before and after washing may affect a hybridization reaction and an interpretation of the reaction results, the state of the spots before washing was identified.

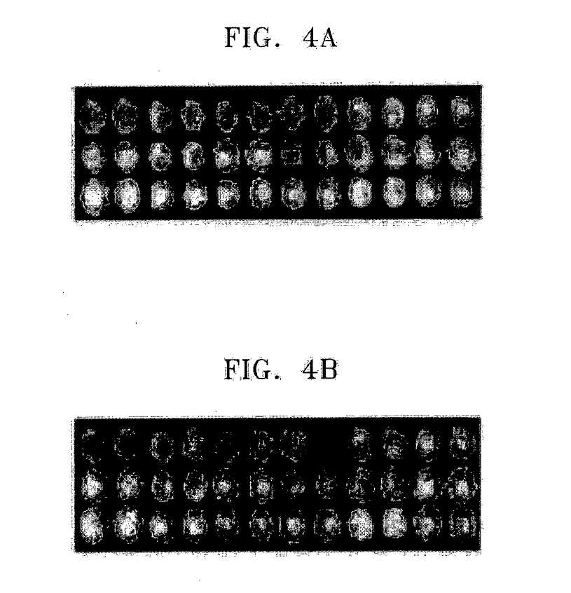

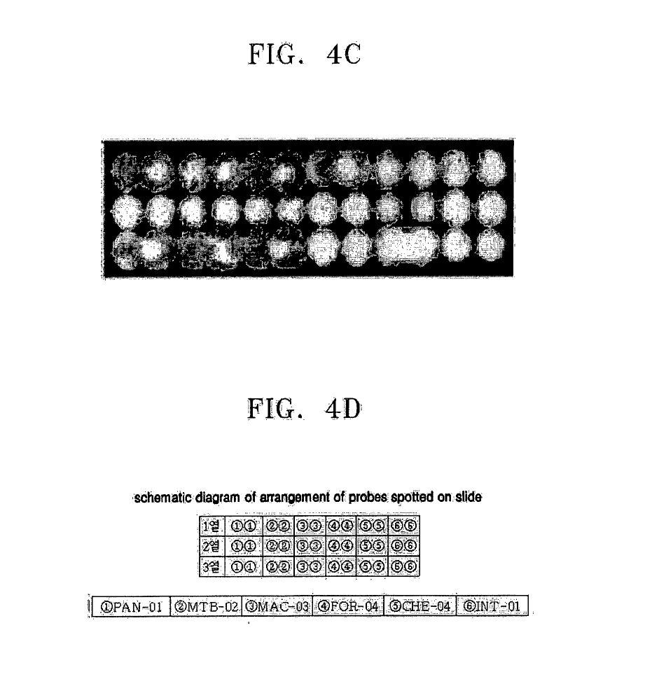

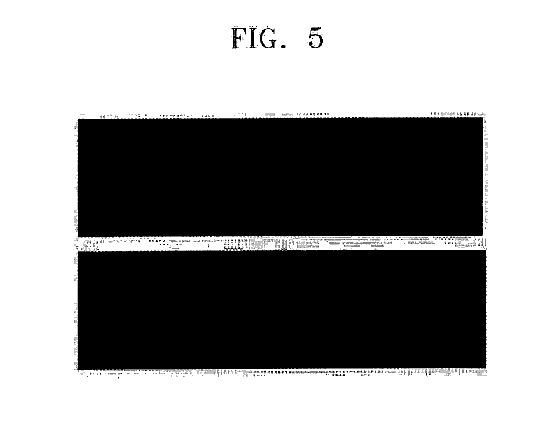

In the microarray fabricated in Example 4, probes were immobilized on the slide as illustrated in FIGS. 4D and 5 and whether the probes were immobilized on the slide and the state of the spots were analysed using a laser scanner.

### Example 7

### Washing of Probes Which Were Not Immobilized

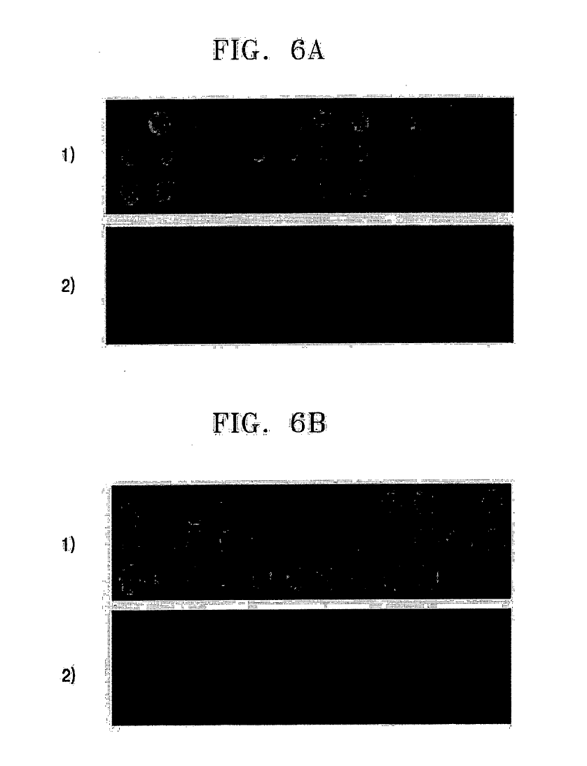

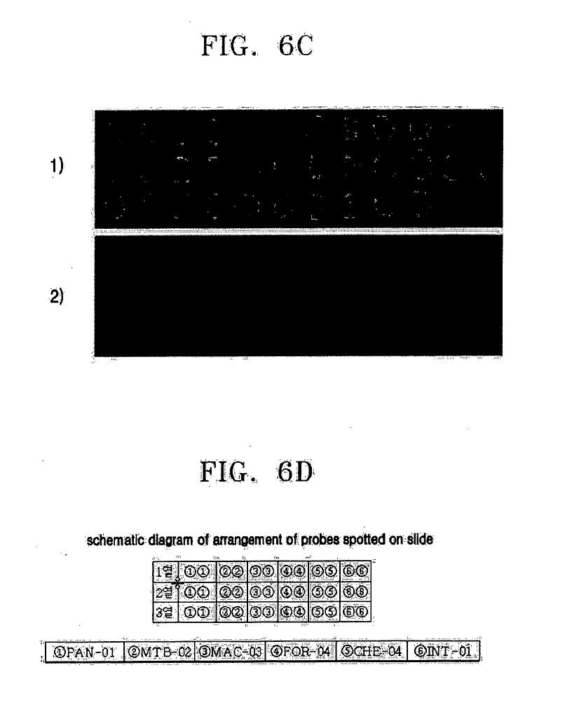

The present Example was performed to wash probes which were not immobilized on the slide processed in Example 4. The slide was washed using a 0.2% SDS diluted solution at room temperature and then washed twice using dH2O. Then, the washed slide was immersed in a solution of sodium borohydride for 5 minutes and washed at 100° C. Finally, the slide was washed with 0.2% SDS and dH2O and thoroughly dried using a centrifuge. Then, the immobilization state of the probe may be analysed with a scanner as illustrated in 1) of FIGS. 6A through 6C to identify washing state.

### Example 8

### Hybridization

The target product labeled with biotin, prepared in Example 5 was heated to be denatured into single strand and cooled to 4° C. 10 μl of a hybridization reaction solution including 1-5 μl of the target product was prepared. The hybridization solution was transferred to the slide processed in Example 7 and covered with a cover slip so as not to produce a bubble. Then, light was blocked and the hybridization reaction solution was reacted at 40° C. for 30 minutes.

### Example 9

### Washing of the Unbounded Target Product

To wash the remaining target products which were not hybridized, the cover slip was removed using a washing solution in which 2× SSC (300 mM NaCl, 30 mM Na-Citrate, pH 7.0) and 0.2% SDS were mixed, and the slide was washed sequentially using a 2× SSC/0.2% SDS, 2× SSC, and 0.2× SSC solution. Finally, the washed slide was completely dried using a centrifuge.

### Example 10

### Binding of Dye and Analysis

To identify whether or not the target product and the target probe were bound, Cy5-steptavidin or Cy3-streptavidin (available from Amersham Pharmacia Biotech, USA) was diluted in 6× SSC and bovine serum albumin (BSA), and then, 40 μl of the solution was transferred to the slide, and then the slide was covered with the cover slip to block light, and then the reaction was performed at 50° C. for about 20 minutes. Since a Cy5-streptavidin dye was used in this Example to identify whether or not the target product and the target probe were bound, the TAMRA fluorescent dye having the same wavelength as that of Cy3 was used to add a fluorescence label to the QC probe.

As described in Example 3, any fluorescent material having a different wavelength from that of the fluorescent material for identifying whether or not the target product and the target probe were bound may be used to label the QC probe. After the reaction, the cover slip was removed with a 2× SSC washing solution and the slide was washed with a 2× SSC and 0.2× SSC solution. To analyze the bonding reaction, the results were analyzed with a laser scanner, GenePix 4000A (available from Axon Instruments, USA) as illustrated in 2) of FIGS. 6A through 6C.

### Example 11

### Preparation of a QC Probe Simultaneously Acting as a Target Probe

A QC probe simultaneously acting as a target probe was synthesized under the same conditions as in Example 3. In the fabrication of the QC probe, to attach the probe to a support, a dT spacer having 5′-Amino-Modifier C6-15 bases and a probe having 15-25 base sequences were synthesized and purified by PAGE. In this Example, Cy5 fluorescent material was used to dye the target product and TMARA fluorescent material was used to label the internal position of the base sequence of the spacer of the target probe. TAMRA having molecular formula of C₂₅H₂₂N₂O₅ can be labeled at 5′-end, 3′-end, and the internal position of the oligonucleotide base sequence The base sequence of the QC probe used in this Example was prepared using the base segment of MTB-10 of SEQ ID NO. 3 in Korean Patent Application No. 2000-0054166 and FOR-06 of SEQ ID NO. 25 in Korean Patent Application No. 2001-0062125 and the prepared probes were as follows.

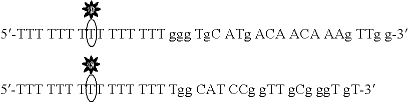

FIG. 1 is a design of a QC probe labeled with a fluorescent material which is used to identify immobilization reactions of probes and illustrates a QC probe directly having a fluorescent material on a base sequence of each target probe or any base sequence on a spacer on a support and a QC probe having a spacer between a base sequence and a fluorescent material. The base sequence of the QC probe of n-mer labeled with the fluorescent material is the same as the base sequence of the target probe or may be any base sequence.

FIG. 2 illustrates the mixed QC probe and target probe immobilized on a support. In FIG. 2, a target probe and a QC probe labeled with a fluorescent material are present together at one spot. The state of the probe such as immobilization, foreign materials, and environmental factors to the probe may be inspected.

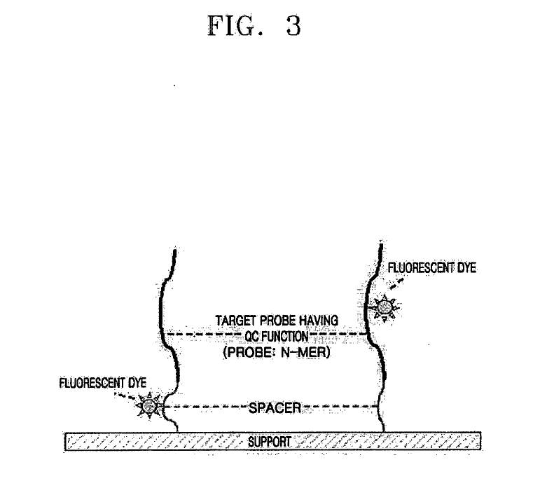

FIG. 3 is a design of a QC probe simultaneously acting as a target probe, in other words, a target probe having a QC function. The QC probe is designed to place a spacer on a support, place a base sequence of each target probe thereon, and locate a fluorescent material on the internal position of the base sequence of the spacer or the target probe.

FIG. 4 shows the results of an analysis of a slide using a scanner after immobilizing a QC probe and a target probe mixed at a certain ratio on the slide and before washing the slide. This analysis is performed to inspect the state of the target probe and whether or not the target probe was immobilized on a support. Apparent from FIG. 4, most of the spots have similar shapes and sizes, but have different sizes according to the type of the support. FIG. 4A shows that the immobilization state, such as shape and size, of probes immobilized on the slide is satisfactory. In FIG. 4B, it is identified that the eighth probe in the first line was not spotted. In FIG. 4C, it is identified that the spotted probes do not have a uniform shape and concentration and several probes are agglomerated due to problems during immobilizing. Although the probes are immobilized in the same quantity on the slide, there are differences in the concentration of the spotted probes after immobilizing. In other words, as a result of the analysis with a scanner, the white spots show an oversaturation state while the green spots show that probes are spotted in a slightly lower concentration than the white spots.

FIG. 5 shows the results of analysis of a slide after immobilizing only a target probe without a QC probe and before washing the slide. Unlike FIG. 4, information regarding each probe spot is not identified due to the absence of the QC probe.

FIG. 6 shows the results of an analysis of a slide with a scanner after immobilizing a QC probe and a target probe on the slide and washing the slide. FIGS. 6A and 6B show the results after a hybridization reaction using the target product of Mycobacterium chelonae and FIG. 6C shows the results of a hybridization reaction of a positive control product to a positive control detection probe in PCR. 1) is the results of an analysis of the slide at a wavelength of 535 nm after washing the slide and before a hybridization reaction, and 2) is the results of an analysis of the slide at a wavelength of 635 nm after a hybridization reaction. Referring to FIG. 6A-1), immobilization, shape and concentration of the QC probe are satisfactory. As seen from FIG. 6A-2), the shape and concentration of the target probe reacted with the target product are satisfactory after a hybridization reaction. FIG. 6B-1) shows the results of the analysis of the slide after washing and before hybridization reaction. As seen from FIG. 6B-1), the first probe in the first line is insufficiently immobilized on the slide to perform a hybridization reaction and many probes in such a state are present on the slide. Also, the ninth probe and the tenth probe in the first and third lines are agglomerated, and thus have poor shapes. In FIG. 6B-2), the same aspect as in FIG. 6B-1) is identified, which may be reflected to interpretation of the result. Referring to FIG. 6C-1), the twelfth probe in the first line was not immobilized on the slide. This aspect may also be identified in the results of analysis of the slide after a hybridization reaction as illustrated in FIG. 6C-2), which may be reflected to interpretation of the result. For example, it may be estimated whether no detection of the probe after a hybridization reaction corresponds to either no occurrence of a hybridization reaction of the target product and the target probe or the result due to problems in immobilization of the probe when fabricating a microarray. Thus, an experiment can be performed excluding microarrays having problems in immobilization of the target probe and the immobilization state and stable reaction conditions can be established by checking problems in the process of immobilizing the probe and washing process.

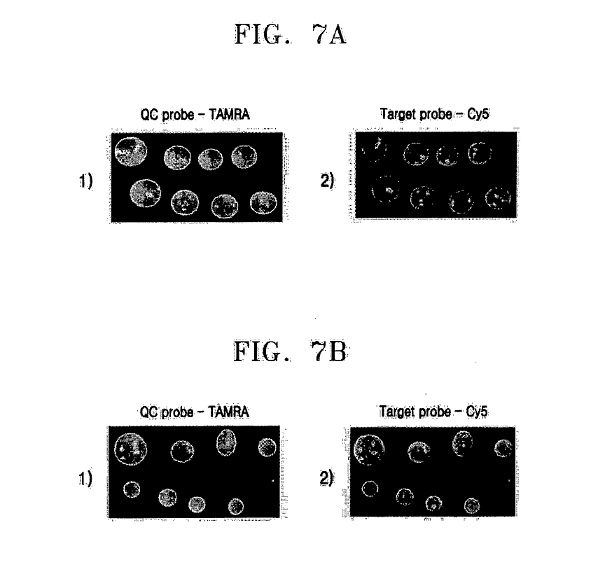

FIG. 7 is the result for the probe prepared in Example 11. In FIGS. 7A and 7B, 1) shows the results of the analysis of probes immobilized on a support after washing and before a hybridization reaction and only a target probe labeled with a fluorescent dye for QC without a separate QC probe is used to identify whether or not probes are immobilized on the support and the immobilization state. FIGS. 7A and 7B, 2) shows the results of the analysis of probes after a hybridization reaction using target products of Mycobacterium tuberculosis and Mycobacterium fortuitum. In FIGS. 7A and 7B, 1) shows the results of the analysis of the probe immobilized on the support after washing and before a hybridization reaction and only a target probe labeled with a fluorescent dye for QC without a separate QC probe was used to accurately identify whether or not probes are immobilized on the support and the immobilization state. As seen from FIGS. 7A and 7B, the shapes and sizes of the spots are different and the concentration of each spot is satisfactory. Referring to FIG. 7A-2) illustrating the results of a hybridization reaction with the target base sequence of Mycobacterium tuberculosis, it can be seen that a hybridization reaction to the target probe of MTB-10 was occurred. Referring to FIG. 7B-2) illustrating the results of a hybridization reaction with the target base sequence of Mycobacterium fortuitum, it can be seen accurately that a hybridization reaction to the target probe of FOR-06 was occurred. As seen from the results of FIG. 7, the target probe labeled with a fluorescent material can act as a QC probe for quality control and can accurately perform a hybridization reaction. Thus, immobilization of the target probe and a hybridization reaction with a target product can be inspected using only the target probe labeled with a fluorescent dye. The target probe labeled with a fluorescent dye can be used to monitor problems in the spotting process of probes and washing process and this monitoring process can be simply performed.

1. A quality control (QC) probe for inspecting a quality of a microarray, in which an oligonucleotide having a complementary sequence to a base sequence of a target product or having any base sequence is labeled with a fluorescent material, the fluorescent material having different excitation/emission wavelength from a fluorescent material labeled in the target product.

2. The QC probe of claim 1, wherein the fluorescent material is labeled at one or more positions of the base sequence of the oligonucleotide and the position is 3′-end, 5′-end, or the internal position of the QC probe.

3. The QC probe of claim 1, wherein a spacer is further included between the probe sequence and the fluorescent material.

4. The QC probe of claim 1, wherein the fluorescent material is at least one material selected from the group consisting of Pyrene, Cyanine 2, GFP, Calcein, FITC, Alexa 488, FAM, Fluorescein Chlorotriazinyl, Fluorescein, Rhodamine 110, Oregon Green, Magnesium Green, Calcium Green, JOE, Cyanine 3, tetramethylrhodamine, TRITC, TAMRA, Rhodamine Phalloidin, Pyronin Y, Lissamine, ROX, Calcium Crimson, Texas Red, Nile Red, Cyanine 5, and Thiadicarbocyanine.

5. The QC probe of claim 1, which acts as a target probe simultaneously by being labeled with a fluorescent material at one or more positions of the base sequence of an oligonucleotide having a complementary sequence to a base sequence of a target product or a spacer base.

6. A method for fabricating a microarray, the method comprising immobilizing the QC probe of any one of claims 1 to 4 and a target probe mixed at a certain ratio on a support of the microarray or immobilizing the QC probe of claim 5 alone on the support of the microarray.

7. The method of claim 6, wherein the QC probe and the target probe are CDNA, oligonucleotide, peptide or protein.

8. The method of claim 6, wherein the QC probe and the target probe are simultaneously immobilized on one spot.

9. A microarray having the QC probe of any one of claims 1 to 4 and a target probe or the QC probe of claim 5 immobilized thereon.

10. The microarray of claim 9, wherein the QC probe and the target probe are simultaneously immobilized on one spot.

11. The microarray of claim 9, wherein the QC probe has the same base sequence as that of the target probe, but is labeled with a fluorescent material having a different excitation/emission wavelength from a fluorescent material for the target probe.

12. A method of inspecting a quality of a microarray, wherein the microarray of claim 9 is used to perform identifying an immobilization state of probes and/or a hybridization reaction with a target product.

13. The method of claim 12, wherein the immobilization state of probes is identified by a scanning fluorescent signal produced by a fluorescent material labeled in a QC probe before or after a hybridization reaction of the target probe and the target product.

14. The method of claim 12, wherein the hybridization reaction of the target probe and the target product is checked by scanning a fluorescent signal produced by a fluorescent material labeled in the target product after a hybridization reaction of the target probe and the target product.

15. The method of claim 12, wherein a probe labeled with fluorescent materials having different wavelengths each other is used to simultaneously inspect an immobilization state of probes and hybridization reaction with a target product.

16. An oligonucleotide including a Mycobacterium genus-specific internal transcribed spacer (ITS) base sequence for differentiating Mycobacteria strains selected from SEQ ID Nos. 1 and 2.
